(19) United States
(12) Patent Application Publication
(10) Pub. No.: US 2019/0390191 A1
(43) Pub. Date: Dec. 26, 2019

(54) CONSTRUCTING ENZYME-BASED SENSORS

(71) Applicant: PharmaSeq, Inc., Monmouth Junction, NJ (US)

(72) Inventor: Wlodek Mandecki, Princeton, NJ (US)

(21) Appl. No.: 16/447,499

(22) Filed: Jun. 20, 2019

Related U.S. Application Data
(60) Provisional application No. 62/687,740, filed on Jun. 20, 2018.

(51) Int. Cl. C12N 15/10

(52) U.S. Cl. CPC C12N 15/1086; C40B 50/06

(57) ABSTRACT

Provided among other things is a method to make a sensor enzyme that comprises: (1) identifying an area on the surface of an enzyme having one to three targeted loops including one linked loop, the loops having 3 or more residues without backbone hydrogen bonding and having up to 10 residues designated as the targeted loop, (2) creating an expression library of more than a thousand recombinant clones expressing recombinant enzyme wherein a segment within one, two or all of the targeted loops are replaced with from three to fourteen amino acid residue segments, (3) performing selection or screening of such library for (a) binding to molecule target substance; (b) enzymatic activity; and (c) allosteric modulation of enzymatic activity by the target substance, and (4) identifying one or more recombinant clones expressing recombinant enzyme that is allosterically modulated by the target substance.

This application claims the priority of U.S. Application No. 62/687,740, filed Jun. 20, 2018.

The present application relates generally to methods of constructing enzyme-based sensors.

### SEQUENCE LISTING STATEMENT

Filed herewith is a Sequence Listing (name: SequenceListing.txt; created: Aug. 15, 2019; sized: 2 KB). The content of that Sequence Listing is incorporated herein by reference in its entirety.

There is a need for robust methods of detecting the presence of chemicals and biomolecules. The chemicals can represent biohazards that are usefully detected in the field. Biomolecules can represent biomarkers for which a rapid test is useful. Such biomarkers can include for example human chorionic gonadotropin, cholesterol, insulin, prostate-specific antigen, and the like.

The inventors have discovered methods of modifying enzymes such they bind to specific substances (that do not bind to the native enzyme), and modified in their enzymatic activity by such binding. As such, the increase or decease in enzyme activity can be used to test for the presence of the chemical in question.

### SUMMARY

Embodiments of the invention include a method to make a sensor enzyme that comprises: (1) identifying an area on the surface of an enzyme having one to three targeted loops including one linked loop, the loops having 3 or more residues without backbone hydrogen bonding and having up to 10 residues designated as the targeted loop, (2) creating an expression library of more than a thousand recombinant clones expressing recombinant enzyme wherein a segment within one, two or all of the targeted loops are replaced with from three to fourteen amino acid residue segments, (3) performing selection or screening of such library for (a) binding to molecule target substance; (b) enzymatic activity; and (c) allosteric modulation of enzymatic activity by the target substance, and (4) identifying one or more recombinant clones expressing recombinant enzyme that is allosterically modulated by the target substance.

### DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only illustrative embodiments of this invention and are therefore not to be considered limiting of its scope, for the invention may admit to other equally effective embodiments.

FIG. 1 depicts 3D structure of an alkaline phosphatase;

FIG. 2 shows the sequence of an alkaline phosphatase;

FIGS. 3A and 3B show targeted loops (defined below) on the surface of a space-filling model of alkaline phosphatase;

FIG. 4 shows a representation of the targeted loops in an alkaline phosphatase example;

FIG. 5 shows illustrative segments of the alkaline phosphatase; and

FIG. 6 shows an exemplary the pJuFo phagemid.

To facilitate understanding, identical reference numerals have been used, where possible, to designate comparable elements that are common to the figures. The figures are not drawn to scale and may be simplified for clarity. It is contemplated that elements and features of one embodiment may be beneficially incorporated in other embodiments without further recitation.

### DETAILED DESCRIPTION

The invention makes use of the extensive date in for example the Protein Data Bank. In 2019, the number of protein structures archived in the Protein Data Bank exceeded 140,000. In excess of 73,000 of these are enzyme structures (source: www.rcsb.org/stats/enzyme). Thus, with the parameters described herein, a great number of these enzymes can be modified to be allosterically modified by binding a given substance (“target substance”).

The invention will be exemplified in detail with respect to a particular “allosterically modified enzyme” (“AME”), alkaline phosphatase. The activity of this enzyme can be monitored directly with color-generating substrates, such as p-nitrophenyl phosphate, from which enzyme activity produces p-nitrophenol (PNP), which is detectable at 410 nm (yellow). Other enzymes may produce a product that in turn is a substrate in a color-producing reaction.

In circumstances where the timing of color production is important, the material prospectively containing the target substance can be contacted with the AME and the substrate. Timing of such contacting can be such that the AME's activity is principally as modified by the target substance (if present).

The method of constructing enzyme-based sensors can be, in embodiments, configured to detect small molecules, namely those molecules of MW of 1,000 or less. In embodiments, the method is configured to detect molecules of MW of 573 or less.

Targeted Loops

Those of skill will recognize surface loops between structured peptide segments in an enzyme three dimensional structure, where the loops include 3 or more residues that do not have consistent backbone hydrogen bonding to other residues in the protein. These loops can be found in the 3D structures archived in the Protein Data Bank, with the determined structure showing the lack of such hydrogen bonding (regardless of short-term hydrogen bonding that may occur during the structural “breathing” of the polypeptide). These loops are typically 3 to 10 residues in length, and in embodiments can include 1 to 3 residues (or 1 to 2 residues, or 1 residue) at each end that can be categorized as in the end portion (amino or carboxy end) of a structured segment, such as without limitation an alpha helix, a similar helix, or beta sheet (parallel or antiparallel). Presence in such a structured element is determined with reference to a 3D structural determination.

To conduct the selection process of the invention, 1 to 3 of such loops within proximity of the enzyme active site are targeted for random insertion/replacement of peptide sequence (“targeted loops”). Preferably 2 to 3 such loops are targeted. The loops that are targeted for such modification or replacement can be termed “modified loops.” Note that all loops designated modified loops are necessarily literally modified, so long as at least one of the loops is modified. Generally it is expected that the linked loop will be modified.

A measure of proximity is that (a) an amino acid whose side chain participates in coordinating a enzyme cofactor (including for example a metal ion) or has been shown to participate in binding substrate or a transition state for the catalyzed reaction is linearly within 1 to 6 amino acids of the nearest end of a said loop (the “linked loop”), and (b) the other loops are sufficiently close to the linked loop that participation in coordinated binding of a “targeted substance” by those of the targeted loops as modified loops is a practical possibility. The practicality of having coordinated binding is measured by having a practical harvest of binding clones in the selection methods described below.

With the proximity of the linked loop to an active component of the active site, the selection methods described below can find a useful harvest of binding sites where binding affects enzyme activity, such as by pulling, pushing or twisting (translation or rotation), or like conformational effects. This physical effect can shift the position of cofactors, or sidechains that form a reaction intermediate or provide hydrogen ion exchange or the like, or entities otherwise contributing to catalysis. As discussed below, this enzyme allosteric effect is systematically screened for, such that a small yield of such activity can nonetheless provide a practical yield.

In embodiments, segments of the targeted loops are replaced with 3 to 14 amino acid segments. In embodiments, segments of the targeted loops are replaced with 3 to 10 amino acid segments.

In embodiments, the targeted loops are 4 to 7 amino acids in length. In embodiments, the targeted loops are 5 amino acids in length. In embodiments, the targeted loops can include up to 2 residues at each end that can be categorized as in the end portion of a structured segment.

In embodiments, only a limited contiguous region of one or more of the targeted loops is subjected to replacement or insertion, such as a 3, 4, or 5-amino acid segment.

Nucleotide Pools; Preferential Use of Grand Catchers

It has been shown that certain amino acids are preferentially useful in forming proteins in binding sites. (Righetti et al. (2010) The proteome buccaneers: how to unearth your treasure chest via combinatorial peptide ligand libraries. Expert Rev Proteomics 7(3):373-385; Bachi et al. (2008) Performance of combinatorial peptide libraries in capturing the low-abundance proteome of red blood cells. 2: Behavior of resins containing individual amino acids. Anal Chem 80(10):3557-3565; Sim6 et al. (2008) Performance of combinatorial peptide libraries in capturing the low-abundance proteome of red blood cells. 1. Behavior of mono- to hexapeptides. Anal Chem 80(10):3547-3556.) These amino acids are Arg (R), His (H), lie (I), Lys (K), Phe (F), Trp (W), Tyr (Y) and Val (V). A somewhat similar list was identified as found in the binding sites for small molecules. These amino acids are Trp, His, Phe, Met, Tyr, Cys, Leu and Asp. (Soga et al., Use of amino acid composition to predict ligand-binding sites. J Chem Inf Model. 2007 47(2):400-406.) It is believed that either one of these lists will provide useful diversity modes of binding, along with a propensity for participating in binding structures.

Combinatorial display and selection methods applied will in embodiments preferentially utilize amino acids of one of these 8 amino acid lists, or amino acids of the 12 amino acids of the combined lists, in the modified loops.

For example, the modified loops can be made with synthetic oligo primer pools using PCR to make matching complementary strands matching segments where during synthesis pooled nucleotides created sequence heterogeneity (that would otherwise be difficult to fully match with a complementary oligonucleotide synthesis). Pooled synthesis strategies for the three positions of a given codon can include for example (with the RNA utilized U, uridine shown, while at the DNA level T, thymine will be used):

| | position 1/position 2/position 3 | Synthesizes |
|---|---|---|
| Codon 1 | U + A pool/U/U + C pool | Ile, Phe, Val |
| Codon 2 | U + C pool/A/U + C pool | His, Tyr |
| Codon 3 | A/A + G pool/A + G pool | Arg, Lys |
| Codon 4 | UGG | Trp |

In embodiments, the modified loops are made from targeted loops by inserting or replacing only with amino acids of one of the above-identified lists, or the combination.

Extensive pools of hundreds of oligonucleotides are from such oligonucleotide houses as Agilent (Santa Clara, Calif.), Twist Biosciences (San Francisco, Calif.), IDT (Coralville, Iowa), Thermo Fisher Scientific (Waltham, Mass.) and others. Different oligo pools will can have different groups of mutations, depending on the synthesis scheme (where pools of activated nucleotides are utilized in a given nucleotide addition step) and on whether different syntheses are pooled.

Complementary DNA strands can be designed with unique overlaps to allow concurrent ligation of several gene segments. For example the strategy can utilize restriction enzymes that cut away from the recognition sequence with an overlap to provide unique annealing sequences. For example, Fokl or Bsal can be used:

| BsaI | FokI |
|---|---|
| 5′...GGTCTC(N)₁^{▾}...3′ | 5′...GGATG(N)₉^{▾}...3′ (SEQ ID NO: 2) |
| 3′...CCAGAG(N)_{5▴}...5′ (SEQ ID NO: 1) | 3′...CCTAC(N)_{13▴}...5′ (SEQ ID NO: 3) |

Methods for such assembly are described for example in Mandecki et al., Gene 1988, 68(1):101-107; and Mandecki et al., Gene 1990, 94(1):103-107.

Randomization based on the grand catcher concept has been tested in another context by Applicant in a project funded by the NIH (grant 1R43CA168014, project titled “Characterization of antitumor antibodies using combinatorial peptide library on RFID p-Chips”). A grand catcher-based four amino acid peptide library was shown to yield peptide binders to antibodies presented as mAbs or in serum. The results are described in detail in the Final Project Report submitted to the NIH.

With two or more of the modified loops providing binding for the target substance, affinity should be higher (due to (a) an additive effect and (b) the binding at one modified loop reducing the entropic barrier to further binding at a second modified loop).

Enzyme Selection

The enzyme is one for which a 3D structure is known in sufficient detail to identify target loops. The specific species of the enzyme may not match that for which detailed structural analysis was conducted, if sequence homology is such that those of skill in protein structure function can identify the target loops.

In embodiments, the enzyme is one with a high Tₘ (temperature at which both the folded and unfolded states are equally populated at equilibrium), such as a Tₘ of about 50° C. or higher, or 60° C. or higher, or 70° C. or higher, or 80° C. or higher, or 97° C. or higher.

In embodiments, the enzyme is one that recovers activity after one or both of heat or acid denaturation, to the extent of 90% or more of activity.

Screening

Expression clones of modified recombinant enzyme are screened for binding the target substance. It is believed that phage display vectors provide the most robust system for testing the numerous clones generated to find clones with binding and a material affect of binding on enzyme activity. This is because of the facility with which positive clones can picked for further workup or screening, and the facility with which the phages can be subjected to selective procedures such as panning. Screening is repeated with every more enriched pools of clones, with perhaps 3 to 5 rounds of screening used. [Please elaborate on how the rounds are conducted]

Screening can be by panning, such as utilizing biotinylated BSA bound to the target substance (“TS”). After incubation of the phage library, which can represent for example 10⁹ or 10¹⁰ clones, with the biotin-BSA-TS, the material is incubated on plates with bound streptavidin. See, e.g., Parmley et al., Gene 73(2), 1988, Pages 305-318. An elution buffer (e.g., 1 mg BSA/ml; 0.1 N HCl, pH adjusted to 2.2 with glycine) is used to elute the binding phages. As indicated above, panning is repeated, even up to 3, 4, 5 or more times.

In embodiments, the phage incubations are conducted in the presence of non-biotinylated BSA, to cut down on selection of BSA-binding clones.

While the initial screenings will pull numerous clones binding the target substance, there can be expected to be false positives, and binders that lack enzyme activity.

The phage vector can be designed to facilitate transfer to an E. coli expression vector. The selected pool can be re-cloned in the E. coli expression vector and plated (e.g., 100,000 clones per plate, 100 plates). Enzyme expression can be tested with an appropriate color generating substrate, or by a secondary color generating reaction dependent on the generation of the enzyme product. For example, for alkaline phosphatase, BCIP (5-bromo-4-chloro-3-indolylphosphate) (Sigma-Aldrich) generates as purple product.

In embodiments, the plates are developed for a period without target substance, and for a period with target substance. This method can capture clones with very weak enzyme activity absent the allosteric effect of the target substance. In other words, clones where the modified loops negatively impact enzyme activity, but target substance binding alleviates that negative effect. Photographs at the endpoints of the two substrate incubations can help identify this effect.

Conversely, in embodiments, the plates are developed for a period with target substance, and for a period without target substance (or without target substance and with antibody to target substance). This method can capture clones with very weak enzyme activity given a negative allosteric effect of the target substance. In other words, clones where binding the modified loops negatively impacts enzyme activity, but reducing target substance concentration alleviates that negative effect. Photographs at the end points of the two substrate incubations can help identify this effect.

Thereafter, individual clones can be grown to provide a crude enzyme product that can be tested for allosteric effect. With modern biotechnology tools, this can be done with a great number of clones. The clones can be grown in microtiter plates, robotically sampled to preserve and archive the bacteria, robotically lysed and optionally centrifuged, and enzyme measurements conducted on the lysate or lysate supernatant. The lysate can be divided to measure activity in the presence and absence of target substance. Thus, sampling of thousands of clones or more is feasible.

Thereafter, bacterial cultures of high performing clones (e.g., 20 clones or more) are grown in higher volume (e.g., 1 liter), and the enzyme partially purified. In embodiments, enzyme purification need not be to homogeneity. For example, differential salt precipitation or the like can obtain an enzyme composition allowing for significant characterization of the enzyme activity. In particular, the k_{cat} and Kₘ can be determined in the presence and absence of target substance. The activity against a range of target substance concentrations indicates how sensitive the enzyme is to the presence of target substance (e.g., IC50 or analog to EC50 for an agonist effect).

Alkaline Phosphatase

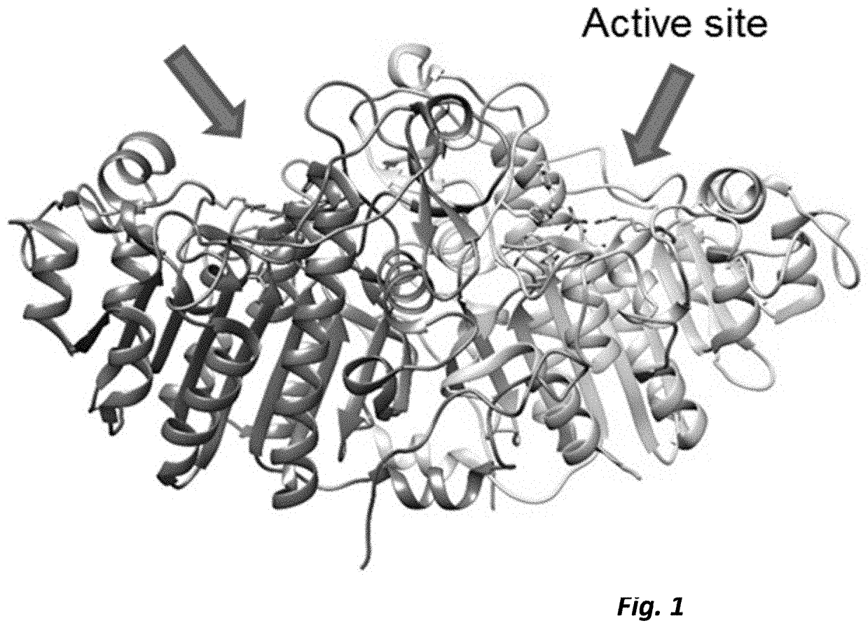

Alkaline phosphatase (ALP) provides a useful illustration of the invention. FIG. 1 shows a ribbon diagram of E. coli as found as the 1alk.pdb structure in the Protein Data Bank (www.rcsb.org). The arrows show the active sites. The structure can be visualized in more detail by going to the Protein Data Bank at www.rcsb.org, typing “1alk” in the search window, and clicking on the 3D View tab. This link allows the user to inspect the structure, including rotating the structure in all 3 dimensions.

Alkaline phosphatase of E. coli (FIG. 1) is a zinc-containing dimeric enzyme with the MW of 86,000 da, each subunit containing 429 amino acids with four cysteine residues linking the two subunits [Coleman JE (1992) Structure and mechanism of alkaline phosphatase. Ann Rev Biophys Biomol Str 21: 441-483]. Alkaline phosphatase contains two Zn ions and one Mg ion per subunit, with Zn occupying active sites A and B, and Mg occupying site C. The mechanism of action of alkaline phosphatase involves the geometric coordination of the substrate between the Zn ions in the active sites. Alkaline phosphatase has a Kₘ of 8.4×10⁻⁴ (for T. aquaticus, [Yeh et al., J Biol Chem 251(10): 3134-3139]). Alkaline phosphatase of E. coli is uncommonly soluble and also is active within elevated temperature conditions, having a Tm of 97° C. [Boulanger et al., J Biol Chem. 2003; 278(26):23497-23501]. Amazingly, the enzyme can be refolded after denaturation [Sykes et al., Proc Natl Acad Sci USA. 1974. 71(2):469-473]. Alkaline phosphatase was cited as the most frequently referenced enzyme [Coleman, Ann Rev Biophys Biomol Str 21: 441-483]. Alkaline phosphatase is encoded by a phoA gene of E. coli. The gene encodes a precursor form of the enzyme that includes the signal peptide that directs the enzyme to the periplasm.

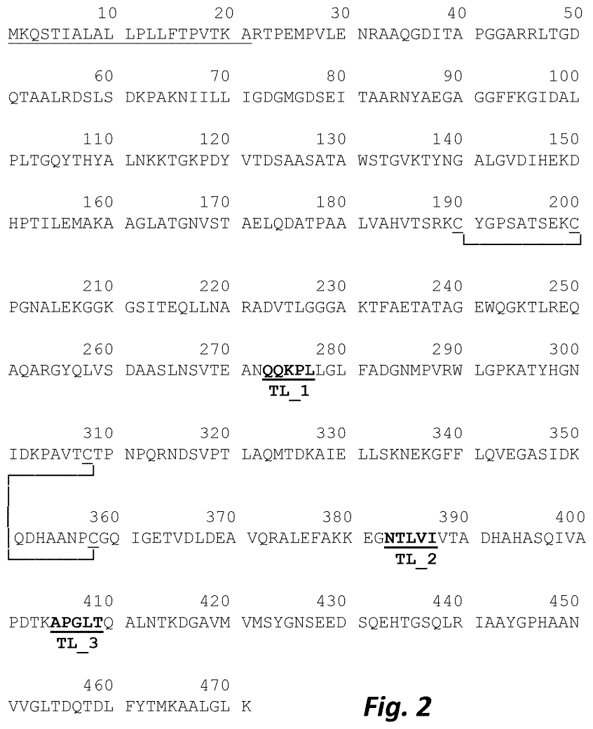

The protein sequence (SEQ ID NO:1) for E. coli ALP is shown in FIG. 2. As shown, the signal peptide is residues 1-21. As such, residue D123 can be numbered in some publications without reference to the signal peptide, such that it is residue D102. Disulfide bonds are C190-C200 and C308-C358. Targeted loops (“TLs”) are TL_1: 273-277, TL_2:383-387 and TL_3:405-409. TL_3 is the linked loop. The TLs can used in making modified loops can extend 1 to 2 residues on one or both ends.

Allosteric regulation by small molecules using the target loop approach has not been demonstrated by others. However, positive ALP regulation by selective monoclonal antibodies to the V3 loop of HIV has been shown. The 13-amino acid V3 loop was inserted between residues 407 and 408. (Brennan, Christianson, La Fleur, Mandecki, Proc Natl Acad Sci USA. 1995 Jun. 20; 92(13):5783-7; Brennan et al., Protein Eng. 1994 April; 7(4):509-14.) Positive regulation implies an effect independent of any possible active site blocking by the antibody.

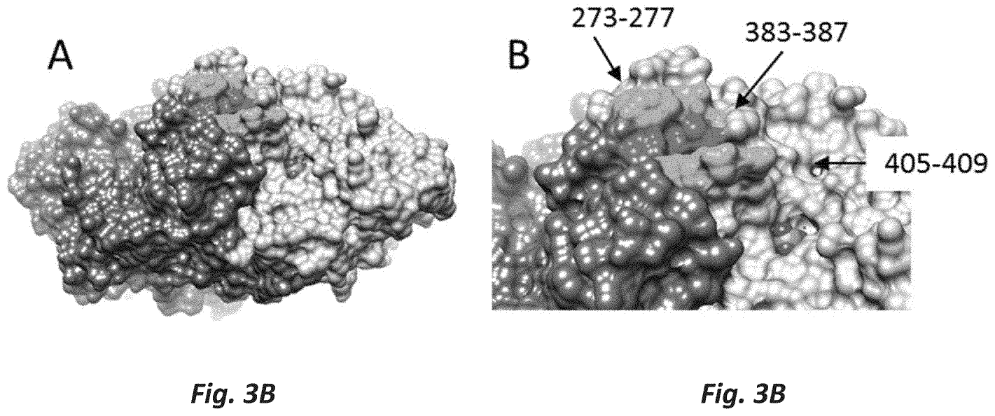

FIGS. 3A and 3B show the TLs on the surface of a space-filling model of ALP. FIG. 3A shows the whole protein, while FIG. 3B is a blown-up segment of interest. Shown in light blue toward the top is TL_1, underneath in violet TL_2, and further underneath in light blue is TL_3. The red shows a phosphate that is indicative of the active site. The binding site area expected to by made from modified loops should thus be about 15 Å by 15 Å.

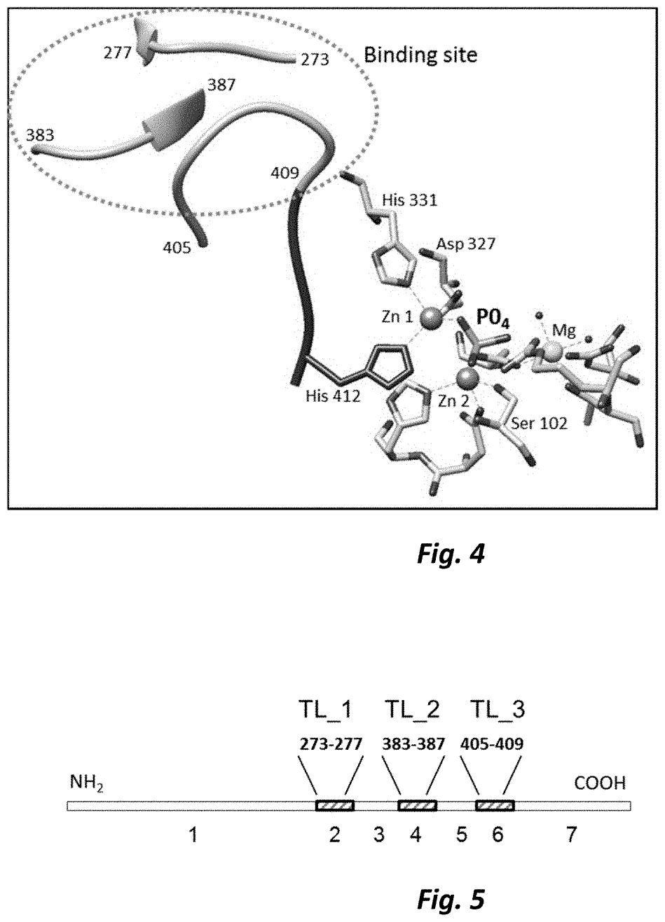

FIG. 4 shows a representation of the targeted loops, their relationship to the active site, and residues involved in coordinating the metal ions and phosphate found in the active site.

FIG. 5 shows illustrative segments of the ALP expressing DNA that can be made by PCR from gene segments or oligonucleotides. Shown are the segments as assembled into the expressing DNA. Those of skill will recognize numerous avenues by which the segments (which may have additional sequence that is removed in assembly) can be joined.

The designed binding site (1-3 modified loops) is located within a subunit of ALP. Knowing that the enzyme is a dimer, there are two binding sites per enzyme. This can be considered an advantage from the perspective of the selection (biopanning). The target substance can be presented as a conjugate to e.g. bovine serum albumin (BSA), with two or more target substances conjugated to a single molecule of BSA. Therefore, a bivalent binding will be possible, where the ALP-carrying phage will be able to bind to two target substances at a time allowing for selection for a higher apparent binding constant.

Exemplary Plasmid Vector; Exemplary Library Construction

A filamentous phage system in which the E. coli alkaline phosphatase is displayed on the tip of the phage, in fusion with the pill coat protein of the phage, can be used. The system has been described by Crameri and his coworkers. [Weichel et al., Open Biochem J. 2008, 2:38-43; Crameri et al., Gene 1993, 137(1):69-75 Crameri et al., Chapter in: “Phage Display In Biotechnology and Drug Discovery”, Second Edition, 2015 CRC Taylor & Francis, New York, N.Y.] If needed, a similar plasmid vector can be synthesized based on publicly available information.

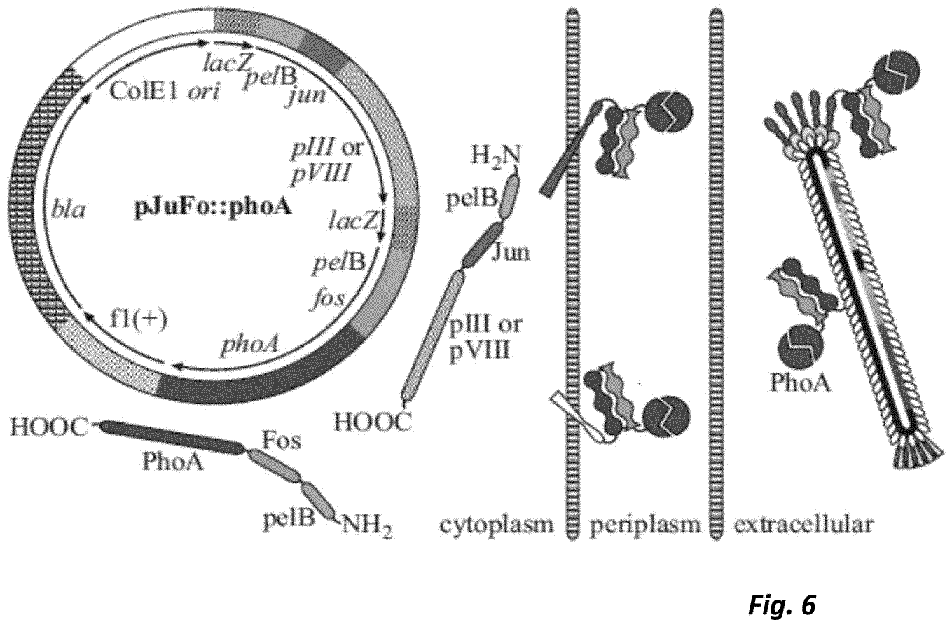

Briefly, the pJuFo phagemid (FIG. 6) contains the origin of replication from filamentous phage, ampicillin resistance, and two constructions related to protein display: (1) phoA gene fused to Fos and (2) phage gpIII fused to Jun (Fos and Jun are two small protein structures, alpha helices, with high propensity to bind to each other, and provide linkage between ALP the phage coat). The pIII-Jun hybrid protein provides an anchor to the phage coat that in turn anchors ALP to the phage coat. To produce ALP-filamentous phage, a superinfection with a helper phage can be used, as described in Mandecki, W., Goldman, E., Sandlie, I. and Løset G. A. (2015) Phage display and selection of protein ligands. In: Practical Handbook of Microbiology, Third Edition (eds. E. Goldman and L. Green), CRC Press, Boca Raton, Fla., part I, chapter 9, 115-134. The helper phage is readily available commercially. A similar vector system can be used with other enzymes.

Since ALP has two identical subunits, the phage producing bacteria can inherently produce, or be transfected to produce, ALP subunits that are not ALP-Fos hybrids, such that only one subunit need be anchored via Fos. Such a product is illustrated in FIG. 6. Alternatively, though not illustrated, both subunits are linked to Fos. Note that the crystal structure of ALP shows that the N-terminal that is linked to Fos extends away from the enzyme, and is on the opposite face from the active site and the target loops. Placement of such binding moieties as Fos with respect to other enzymes can be made with respect to similar considerations. The highly favorable location used in this example is not a requirement for all embodiments.

As illustrated in FIG. 6, the two expressed proteins can be expressed with a leader sequence (e.g. pelB) that directs the expressed protein to the periplasm (between the two membranes of gram negative bacteria). The leader sequence is typically removed during the membrane transport process.

The phoA gene expression segment and the mutated regions are schematically presented in FIG. 5. All of the illustrative mutations are in the region corresponding to amino acids 273 to 409, which is 411 bp long. This fragment will be made by PCR from synthetic oligonucleotides. A pool of more than 10¹⁰ different DNAs are made in a series of PCR reactions.

The gene is subdivided into seven fragments, three of which (fragments 2, 4 and 6 in FIG. 5) are subjected to mutagenesis. The fragments coding for the constant regions (1, 3, 5 and 7) are made by simple PCR from the parent vector pJuFo::phoA (FIG. 8). Eight custom oligo primers will be needed for that synthesis of the constant regions. The fragments coding for the variable regions 2, 4, and 6 will be made by PCR from above described oligo pools (e.g., 3 oligo pools). Six additional custom oligo primers will be needed. All primers carry (e.g.) the Bsal restriction site. This site allows for the creation of unique 4 nt long 5′ protruding ends outside the enzyme's recognition DNA sequence and is, therefore, convenient for cloning. Each of the seven DNA fragments obtained will be digested with Bsal for assembly by annealing and ligation reactions.

All the fragments are isolated on a gel in preparative quantities. The seven fragments are ligated in a single ligation analogous to the Fokl gene synthesis method described in the above-cited Mandecki et al. papers to create a full-length mutated phoA gene (comprising a pool of mutants) that is then cloned into the pJuFo::phoA vector (FIG. 6). Cloning and transformation into E. coli is done at a scale allowing for the making of 10⁶-10¹⁰ independent clones.

Selection of sensor ALP specific to a given small molecule is done in three phases. Exemplary target substances (small molecules) include:

TABLE
1. Biotin (MW of 244 da)
2. FITC (389 da)
3. 17β estradiol (272 da)
4. Neuropeptide Met-enkephalin (Tyr-Gly-Gly-Phe-Met) (573 da)
5. Thyrotropin-releasing hormone (TRH; 362 da)

First, in Selection 1, those phages that bind to the target substance are selected. The small molecule is presented as a conjugate to bovine serum albumin (BSA), which is commonly done when selecting phage-based antibodies for binding to small molecules [e.g., Schofield et al., Genome Biol. 2007, 8(11):R254]. The ALP phage library will go through a panning procedure previously described for antibodies [e.g., Schofield et al.]. Three or four rounds of panning are conducted. The obtained pool of phage clones contains a large number of binders. A number, even a large number, are false positives, and another group of clones do not have ALP activity. However, these non-functional clones are systematically excluded by the further methods described herein.

Second (Selection 2), the clones that have ALP activity are selected. The gene library after Selection 1 is re-cloned into an expression plasmid. The expression plasmid library is used to transform E. coli, which are plated on plates (Petri dishes) containing the agar-based medium that allows for color identification of clones producing alk phos. This is based on color conversion of BCIP (5-bromo-4-chloro-3-indolylphosphate) (Sigma-Aldrich) to purple by ALP. This step is used for screening of 10⁵ clones (10,000 colonies per plate, on 100 plates) or more. All colonies capable of color conversion are harvested.

Third (Selection/Screening 3), clones making enzyme whose enzymatic activity can be either increased or decreased in the presence of the target substance, are identified. Individual clones from the second step are grown in a liquid growth medium, and a crude or partially purified extract is prepared. ALP activity is measured with or without the target substance in the assay.

High performing clones are characterized further.

Polypeptide Sequences for AMEs

Reference Sequence 1 is an enzyme polypeptide sequence less any leader or targeting sequence that is processed away during the cellular protein synthesis process.

Reference Sequence 2 is Reference Sequence 2 minus any targeted loops (typically up to 18 amino acid residues).

Polypeptide embodiments further include an isolated polypeptide comprising a polypeptide having at least a 50, 60, 70, 80, 85, 90, 95, 97 or 98% identity (homology) to a polypeptide reference sequence of Reference Sequence 1 or 2, wherein said polypeptide sequence may be identical to the reference sequence of Reference Sequence 1 or 2 or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids in Reference Sequence 1 or 2 by the integer defining the percent identity divided by 100 and then subtracting that product from said total number of amino acids in Reference Sequence 1 or 2, or: nₐ≤xₐ−xₐ·y), wherein nₐ is the number of amino acid alterations, xₐ is the total number of amino acids in Reference Sequence 1 or 2, y is 0.50 for 50%, 0.60 for 60%, 0.70 for 70%, 0.80 for 80%, 0.85 for 85%, 0.90 for 90%, 0.95 for 95%, 0.97 for 97% or 0.97 for 98%, and is the symbol for the multiplication operator, and wherein any non-integer product of xₐ and y is rounded down to the nearest integer prior to subtracting it from xₐ.

Specific embodiments according to the methods of the present invention will now be described in the following examples. The examples are illustrative only, and are not intended to limit the remainder of the disclosure in any way.

All ranges recited herein include ranges therebetween, and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4 or more, or 3.1 or more. If there are two ranges mentioned, such as about 1 to 10 and about 2 to 5, those of skill will recognize that the implied ranges of 1 to 5 and 2 to 10 are within the invention.

Where a sentence states that its subject is found in embodiments, or in certain embodiments, or in the like, it is applicable to any embodiment in which the subject matter can be logically applied.

The patent or application file contains at least one drawing executed in color. Copies of this patent or patent application publication with color drawing(s) will be provided by the Office upon request and payment of the necessary fee.

This invention described herein is of a method of making a sensor enzyme. Although some embodiments have been discussed above, other implementations and applications are also within the scope of the following claims. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims. More specifically, those of skill will recognize that any embodiment described herein that those of skill would recognize could advantageously have a sub-feature of another embodiment, is described as having that sub-feature

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

The invention can be further defined with reference to the following numbered embodiments:

### Embodiment 1

A method to make a sensor enzyme that comprises: (A) identifying an area on the surface of an enzyme located in proximity of the active site, such an area having a potential impact on the enzyme activity, (B), choosing from one, two or three loops in said area within which up to ten amino acid residues are replaced with from three to ten amino acid residues from a group of all possible (twenty) encoded amino acids, or from a subset of amino acids, such as a “grand catcher” group of eight amino acids, (C) creating an expression library of more than a thousand recombinant clones (genes) expressing recombinant enzyme having a diversity of said replacement sequences in one of many forms (phage display, ribosomal display, yeast display, or the like), (D) performing selection or screening of such library or derivative of such library (such as a panned selection of clones re-cloned in a new expression vector) for (a) binding to molecule target substance; (b) enzymatic activity; and (c) allosteric modulation of enzymatic activity by the target substance (where in embodiments selection or testing of allosteric modulation can comprise testing for binding, activity and allosteric modulation), and (E) identifying one or more recombinant clones expressing recombinant enzyme that is allosterically modulated by the target substance.

### Embodiment 2

A method to make a sensor enzyme that comprises: (1) identifying an area on the surface of an enzyme having one to three targeted loops including one linked loop, the loops having 3 or more residues without backbone hydrogen bonding and having up to 10 residues designated as the targeted loop, (2) creating an expression library of more than a thousand recombinant clones expressing recombinant enzyme wherein a segment within one, two or all of the targeted loops are replaced with from three to fourteen amino acid residue segments, (3) performing selection or screening of such library for (a) binding to a target substance; (b) enzymatic activity; and (c) allosteric modulation of enzymatic activity by the target substance, and (4) identifying one or more recombinant clones expressing recombinant enzyme that is allosterically modulated by the target substance.

### Embodiment 3

The method of a numbered method to make Embodiment, wherein selection or screening comprises one or more selections or screenings effective to show binding and allosteric modulation of enzymatic activity.

### Embodiment 4

The method of a numbered method to make Embodiment, wherein the expression library expresses enzyme having 90% (or 95%, 97% or 98%) sequence identity with Reference Sequence 1 or 2.

### Embodiment 5

The method of a numbered method to make Embodiment, wherein in any modified targeted loop a segment of 5 amino acids is replaced. (As in all embodiments, the diverse replacement segments may include some elements that may mirror elements of the original. The diverse replacements may of course very occasionally match the original sequence. Where only such a targeted loops is modified, this is an ineffective clone that is screened out during the process.)

### Embodiment 6

The method of a numbered method to make Embodiment, wherein in any modified targeted loop a segment of 4 amino acids is replaced.

### Embodiment 7

The method of a numbered method to make Embodiment, wherein in any modified targeted loop a segment of 3 amino acids is replaced.

### Embodiment 8

The method of a numbered method to make Embodiment, wherein any replaced segment is replaced by a segment of equal length.

### Embodiment 9

The method of a numbered method to make Embodiment, wherein the target substance is a small molecule.

### Embodiment 10

The method of a numbered method to make Embodiment, wherein the enzyme is ALP.

### Embodiment 11

The method of Embodiment 10, wherein the expression library expresses enzyme having 90% (or 95%, 97% or 98%) sequence identity with Reference Sequence 1 or 2.

### Embodiment 12

An ALP polypeptide having 90% sequence identity with Reference Sequence 1 or 2, but differing from Ref. Seq. 1 within one or more of the following segments: TL_1, TL_2 or TL_3, and having alkaline phosphatase activity, wherein if differences are in TL_3, a differing TL_3 comprises 3 to 10 amino acids

### Embodiment 13

The polypeptide of Embodiment 11, wherein any differing TL_1 or TL_2 segments comprise 3 to 10 amino acids.

### Embodiment 14

A nucleic acid expression library of clones expressing 1,000 or more distinct polypeptides according to Embodiment 12 or 13.

### Embodiment 15

Enzyme sensor made using a method to make Embodiment above.

### Embodiment 16

Kits for multiplex assays comprising the enzyme sensor and reagents for a sensor assay.

What is claimed is:

1. A method to make a sensor enzyme that comprises:
identifying an area on the surface of an enzyme having one to three targeted loops including one linked loop, the loops having 3 or more residues without backbone hydrogen bonding and having up to 10 residues designated as the targeted loop,
creating an expression library of more than a thousand recombinant clones expressing recombinant enzyme wherein a segment within one, two or all of the targeted loops are replaced with from three to fourteen amino acid residue segments,
performing selection or screening of such library for (a) binding to molecule target substance; (b) enzymatic activity; and (c) allosteric modulation of enzymatic activity by the target substance, and
identifying one or more recombinant clones expressing recombinant enzyme that is allosterically modulated by the target substance.

2. The method of claim 1, wherein selection or screening comprises one or more selections or screenings effective to show binding and allosteric modulation of enzymatic activity.

3. The method of claim 1, wherein the expression library expresses enzyme having 90% sequence identity with Reference Sequence 1 or 2.

4. The method of claim 1, wherein in any modified targeted loop a segment of 5 amino acids is replaced.

5. The method of claim 1, wherein in any modified targeted loop a segment of 4 amino acids is replaced.

6. The method of claim 1, wherein in any modified targeted loop a segment of 3 amino acids is replaced.

7. The method of claim 1, wherein any replaced segment is replaced by a segment of equal length.

8. The method of claim 1, wherein the enzyme is ALP.

9. The method of claim 8, wherein the expression library expresses enzyme having 90% sequence identity with Reference Sequence 1 or 2.

10. The method of claim 1, wherein the target substance is a small molecule.


SEQUENCE LISTING (3 sequences)

SEQ ID NO: 1 - DNA, 11 bp, Artificial
  Restriction Recognition Sequence
  misc_feature (1)..(5): n is a, c, g, or t
nnnnngagacc

SEQ ID NO: 2 - DNA, 14 bp, Artificial
  Restriction Recognition Sequence
  misc_feature (6)..(14): n is a, c, g, or t
ggatgnnnnnnnnn

SEQ ID NO: 3 - DNA, 18 bp, Artificial
  Restriction Recognition Sequence
  misc_feature (1)..(13): n is a, c, g, or t
nnnnnnnnnnnnncatcc